(19) United States
(12) Patent Application Publication
(10) Pub. No.: US 2022/0205795 A1
(43) Pub. Date: Jun. 30, 2022

(54) INFORMATION PROCESSING APPARATUS, INFORMATION PROCESSING METHOD, AND NON-TRANSITRY STORAGE MEDIUM

(71) Applicant: TOYOTA JIDOSHA KABUSHIKI KAISHA, Toyota-shi (JP)

(72) Inventors: Toyokazu NAKASHIMA, Nagoya-shi (JP); Makoto TAMURA, Miyoshi-shi (JP); Yusuke MITOMA, Nisshin-shi (JP); Kosuke KOTAKE, Nagoya-shi (JP); Junko KATOH, Nagoya-shi (JP); Noriko KOREYASU, Toyota-shi (JP); Hiromi TONEGAWA, Konan-shi (JP); Aiko FUJII, Nagoya-shi (JP); Yuutarou MIYASHITA, Nagoya-shi (JP)

(73) Assignee: TOYOTA JIDOSHA KABUSHIKI KAISHA, Toyota-shi (JP)

(21) Appl. No.: 17/456,610

(22) Filed: Nov. 26, 2021

(30) Foreign Application Priority Data
Dec. 24, 2020 (JP) 2020-215042

(51) Int. Cl. G01C 21/34; G06Q 10/02

(52) U.S. Cl. CPC G01C 21/3438; G06Q 10/02; G01C 21/3453

(57) ABSTRACT

An information processing apparatus includes a controller that acquires travel information including a planned travel route and a travel schedule of a moving body on which a rescuee rides, acquires position information of one or more rescuer candidates capable of rescuing the rescuee, and determines a rescuer from among the one or more rescuer candidates based on the position information of the one or more rescuer candidates and the travel information and determines a meeting point at which the rescuer should meet the rescuee.

### CROSS REFERENCE TO THE RELATED APPLICATION

This application claims the benefit of Japanese Patent Application No. 2020-215042, filed on Dec. 24, 2020, which is hereby incorporated by reference herein in its entirety.

### BACKGROUND

### 1. Technical Field

The present disclosure relates to an information processing apparatus, an information processing method, and a non-transitory storage medium.

### 2. Description of Related Art

Conventionally, there is a critical care assist system including a user terminal device, a third-party terminal device, and a critical care assist device. This critical care assist system determines the urgency of critical care based on the biometric information of the user. In addition, the assist system calculates the distance information between the user and a plurality of third parties. Then, the critical care assist system transmits lifesaving instruction information based on the urgency of the critical care, the distance information, and the attribute information of the third parties (for example, Japanese Patent Application Publication No. 2012-222443).

### SUMMARY

An object of the present disclosure is to provide an information processing apparatus, an information processing method, and a non-transitory storage medium capable of determining a rescuer and a rescue point suitable for rescuing a rescuee who is traveling.

One aspect of the present disclosure is an information processing apparatus. This information processing apparatus has a controller that acquires travel information including a planned travel route and a travel schedule of a moving body on which a rescuee rides, acquires position information of one or more rescuer candidates capable of rescuing the rescuee, and selects the rescuer from the one or more rescuer candidates and determines a meeting point at which a rescuer should meet the rescuee.

An aspect of the present disclosure may include at least one of an information processing method, an information processing system, and a non-transitory storage medium for storing a program that have the same characteristics as the information processing apparatus.

According to the present disclosure, it is possible to determine a rescuer and a rescue point that are suitable for rescuing the rescuee who is traveling.

### BRIEF DESCRIPTION OF THE DRAWINGS

FIG. 1 illustrates an example of an information processing system according to an embodiment;

FIG. 2 illustrates an example of the structure of a server according to the embodiment;

FIG. 3 illustrates an example of the data structure of a vehicle database;

FIG. 4 illustrates an example of the data structure of a rescuer database;

FIG. 5 illustrates an example of the structure of a terminal device 30 that can be used as a terminal 3 and a terminal 4;

FIG. 6 is a flowchart illustrating an example of processing by a terminal of a moving body;

FIG. 7A illustrates an example of the format of a message for an emergency request;

FIG. 7B illustrates an example of the format of a message for notification;

FIG. 8 is a flowchart illustrating an example of processing by the server;

FIG. 9 is a flowchart illustrating an example of determination of a rescuer and a meeting point; and

FIG. 10 is a flowchart illustrating an example of processing by a terminal of a rescuer.

### DESCRIPTION OF EMBODIMENTS

An information processing apparatus according to an embodiment includes a controller. The controller performs the following:
(1) Acquiring travel information that includes the planned travel route and the travel schedule of a moving body on which the rescuee rides.
(2) Acquiring the position information of one or more rescuer candidates capable of rescuing the rescuee and determining a rescuer from among the one or more rescuer candidates based on the travel information and the position information of the one or more rescuer candidates.

According to the information processing apparatus, the rescuer can perform rescue at a timing that matches the travel schedule of the moving body based on the travel information and the position information of the one or more rescuer candidates.

Here, the moving body is an object on which a person rides to travel. The term “moving body” may include a bicycle, a motorcycle, a vehicle, a railroad vehicle, a ship, an aircraft, and the like. The type of the moving body and the type of the driving mechanism do not matter. The information indicating the planned travel route indicates a route in which the moving body travels and indicates, for example, a course in which a train, which is the moving body, travels from the start point to the end point through a plurality of stations. Alternatively, the information indicating the planned travel route indicates a course in which a bus, which is the moving body, travels from the start point to the end point through a plurality of bus stops. The travel schedule indicates the times (planned arrival time and planned departure time) when a train or a bus stops at stations or bus stops in the example described above. However, the information indicating the planned travel route and the travel schedule is not limited to the example described above.

Here, the rescuee is a person who is unable or difficult to autonomously act during travel by the moving body and needs rescue by the rescuer for some reason. For example, the rescuee includes a person who is sick or injured in or on the moving body for some reason (such as an accident or trouble). In addition, the rescuee may include a person whose sickness or injury conditions become worse during travel by the moving body (such as hypoglycemia) and a person who needs rescue by the rescuer for some reason (anemia, dizziness, palpitation, other poor health conditions, or pregnancy).

The rescuee is not necessarily a person (person who needs transportation by an ambulance) who needs emergency treatment. The rescuee includes a person who should be stopped from travel by the moving body. For example, a person who has a chronic illness or an underlying illness and will recover naturally if getting off the moving body and taking a rest, but becomes worse conditions thereof if continuing to travel by the moving body.

The rescuer is a person who performs rescue actions on the rescuee. The actions may include medical actions (actions permitted only for doctors) and actions (lifesaving actions such as artificial respiration or chest compressions, treatments of abrasions, and the like) that can be performed by a person other than doctors. In addition, the actions may include care actions (such as assistance or support for walking of the rescuee or transportation of the rescuee), attendance, or watching (appropriate observation of or response to changes in conditions). An action on the rescuer requires qualification for taking the action depending on the action, but the rescuer is not necessarily required to be a qualified person.

When the rescuee gets off the moving body, the person who should essentially take care of the rescuee may not be present or the person may not be able to support the rescuee immediately. For example, this applies the case in which there is no person because the drop-off place is an unmanned station or a bus stop or the case in which the driver and the conductor are not on board because the moving body operates automatically. Alternatively, this also applies the case in which it takes time for an ambulance to arrive after drop-off, the case in which a shortage of station staff makes immediate attendance difficult, the case in which the disease condition of the rescuee is not enough to request the dispatch of an ambulance, or the case in which the rescuee wants to avoid requesting rescue from the ambulance crew or station staff because it is exaggerated, but the rescuee wants help from other persons. The rescuer gives a necessary act such as treatment of minor injury, support for walking, or transportation to the rescuee who has gotten off the moving body, as well as watching or attendance, which is the minimum act. As described above, the rescuer other than the administrator (transportation operator) of the moving body such as station staff, the driver of the moving body, and the ambulance crew of an ambulance rescues the rescuee, thereby enabling protection of the health and safety of the rescuee who is on the moving body.

The rescuer may rescue the rescuee in cooperation or collaboration with the administrator (such as the station staff, the driver, or the conductor) of the moving body or the ambulance crew of an ambulance, or may perform rescue in place of or independently of the administrator and the ambulance crew. That is, the rescuer may perform rescue in place of the station staff or the like by receiving a request from the administrator of the moving body. Alternatively, the rescuer may rescue the rescuee who does not need the dispatch of an ambulance. Alternatively, the administrator (administrative organization) of the rescuer concludes a rescue contract in advance with a person who can be the rescuee (for example, a single person with an underlying disease or chronic illness) and the rescuer may come to rescue by receiving contact from the rescuee.

The information processing apparatus may adopt the following structure. That is, the information processing apparatus may adopt the structure in which the controller selects the rescuers from the rescuer candidates who are present in a predetermined geographical range from a planned travel route of the moving body. By selecting one of the rescuers that can meet the rescuee as soon as possible, the timing of meeting can become earlier. In addition, by limiting the rescuer candidates to those who are present in the geographical range, the processing load for calculating the rescuer can be reduced. In other words, it is possible to determine the rescuer and the meeting point that make the time from when the rescuer is requested for rescue until the rescuer and the rescuee meet the shortest. This can determine the rescuer and the rescue point (meeting point) suitable for rescuing the rescuee during travel such as an emergency patient in the moving body.

The information processing apparatus may also adopt the following structure. That is, the controller assumes the point and the time at which each of the rescuer candidates can meet the rescuee in the shortest time period based on the travel information and the position information of each of the rescuer candidates and determines the rescuer in order of the time from earliest to latest. This can make the meeting timing between the rescuer and the rescuee earlier.

The information processing apparatus may also adopt the following structure. That is, the controller determines the rescuer by preferentially selecting the rescuer candidate of the same sex as the rescuee when there are a plurality of rescuer candidates for whom the time at which the rescuer candidates meet the rescuee is the earliest. The number of the rescuers for the rescuee may be one or more than one. However, rescuer candidates more than a predetermined number of rescuers may be extracted. In this case, by preferentially selecting the rescuer of the same sex as the rescuee, the rescuee can easily accept the rescuer who is another person. However, one of the sexes (male and female) of the rescuer may be preferentially selected based on information indicating the desired sex of at least one of the rescuee and the rescuer.

The information processing apparatus may also adopt the following structure. That is, the controller determines the rescuer based on the information indicating the rescue actions to be performed on the rescuee and the information indicating the actions that can be performed on the rescuee when there are a plurality of rescuer candidates for whom the time at which the rescuer candidates meet the rescuee is the earliest. Depending on the situations of the rescuee, the action of the rescuer may be merely attendance and the rescuee may not require treatment by a doctor or a nurse. In this case, when a doctor or a nurse is determined as the rescuer, if another rescuee that requires a doctor or a nurse appears, the doctor or the nurse may be excluded from the rescuer candidate. When the structure described above is adopted, the rescuer can be selected according to the action to be performed on the rescuee.

The information processing apparatus may also adopt the following structure. That is, the controller extracts points in the planned travel route at which the moving body can stop and determines the meeting point among the extracted points. This enables the rescuer to meet the rescuee at a point at which the rescuee can get off safely. The point at which the moving body can stop is, for example, a station for trains or the like or a bus stop. However, the point may include a place other than a station and a bus stop. For example, the point may include a garage or a base. When the moving body is a vehicle, the point may be a parking space, a square (vacant lot), or a park at which the rescuee can safely get off.

The information processing apparatus may also adopt the following structure. That is, the controller notifies the moving body and the rescuer of the meeting point. This enables the moving body to know the place in which the rescuee gets off and the rescuer to know the meeting point.

The information processing apparatus, the information processing method, and the non-transitory storage medium according to the embodiment will be described below with reference to the drawings. The structure of the embodiment is an example and the present disclosure is not limited to the structure of the embodiment.

Structure of an Information Processing System

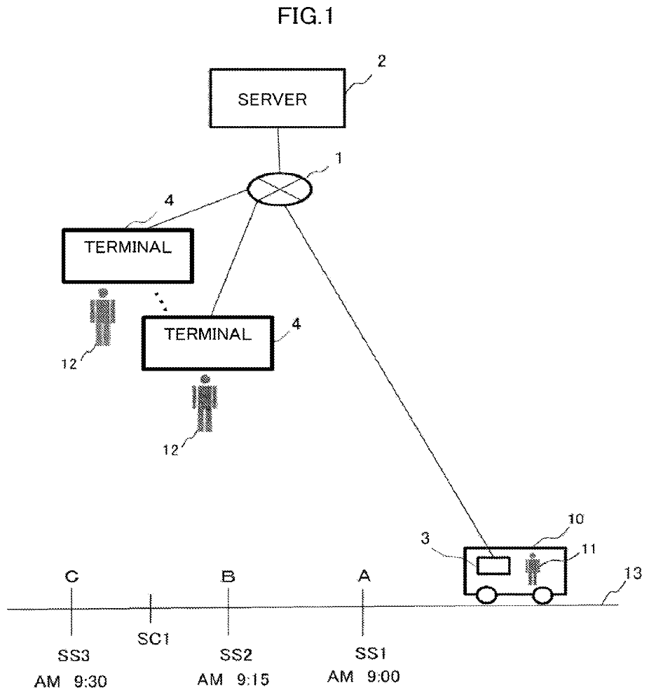

FIG. 1 illustrates an example of an information processing system according to the embodiment. In FIG. 1, the information processing system includes a network 1, a server 2, a terminal 3, and a terminal 4 that are connected to the network 1.

The network 1 is, for example, a public telecommunication network such as the Internet, and can be a wide area network (WAN) or other communication networks. The network 1 may include cellular networks such as Long Term Evolution (LTE) and 5G or wireless networks (wireless route) such as a wireless LAN (local area network including Wi-Fi) and BLE.

The terminal 3 is a terminal installed in a railroad vehicle 10, which is an example of the moving body, and can communicate with the server 2 via the network 1. The terminal 3 may be an in-vehicle terminal of the railroad vehicle 10 or may be a terminal held (carried) by the driver or the conductor of the railroad vehicle 10. Alternatively, the terminal 3 may be held by a passenger (person 11) of the railroad vehicle 10 who becomes the rescuee.

The railroad vehicle 10 travels on a railroad 13 from the start point to the end point. There are stops A, B, and C between the start point and the end point. The part of the railroad 13 from the current position of the railroad vehicle 10 to the end point is an example of a planned travel route of the moving body. The stops A, B, and C are planned stop positions SS1, SS2, and SS3 of the railroad vehicle 10. It is assumed that the planned arrival time of the railroad vehicle at station A is 9 a.m. and the railroad vehicle 10 will depart for station B a few minutes later. The planned arrival time of the railroad vehicle 10 at station B is 9:15 a.m. and the railroad vehicle 10 will depart for station C a few minutes later. The planned arrival time at station C is 9:30 a.m. and the railroad vehicle 10 will depart a few minutes later. The planned arrival and departure times at stations A, B, and C are an example of the travel schedule of the moving body.

In addition, there is a stoppable position SC1, which is not a station, between station B and station C. The stoppable position SC1 is a base or a garage, but may be a place other than these. It should be noted that the railroad vehicle 10 may be a vehicle (for example, a bus), stations A, B, and C may be bus stops, and the stoppable position SC1 may be a garage. As described above, the position at which the moving body can stop may be a place (position) other than a station or a bus stop. When use of the stoppable position SC1 is determined, the planned arrival time at this position will be calculated.

The terminal 4 is used by each of persons 12 who are rescuer candidates. The terminal 4 may be a mobile terminal (wireless terminal) or a fixed terminal. The persons 12 can go to a planned stop position or a stoppable position in response to a request, meet the rescuee who has gotten off the railroad vehicle 10, and become the rescuers (that is, the rescuer candidates) for the rescuee. The rescuer candidates are preferably persons who have knowledge, experience, or qualification as the rescuers, such as a doctor, a nurse, a caregiver, an ambulance crew, and a person who has received a training course for emergency lifesaving.

The terminal 3 transmits a message for a rescue request to the server 2 when the person 11 needs to be rescued within the railroad vehicle 10 that is traveling. When receiving the message for a rescue request, the server 2 determines the person who becomes the rescuer among the persons 12 who are the rescuer candidates and determines the meeting point at which the rescuee (person 11) meets the determined rescuer (person 12). The server 2 transmits the message for notification including the information indicating the meeting point to the terminal 4 and the terminal 3 (railroad vehicle 10).

Structure of the Server

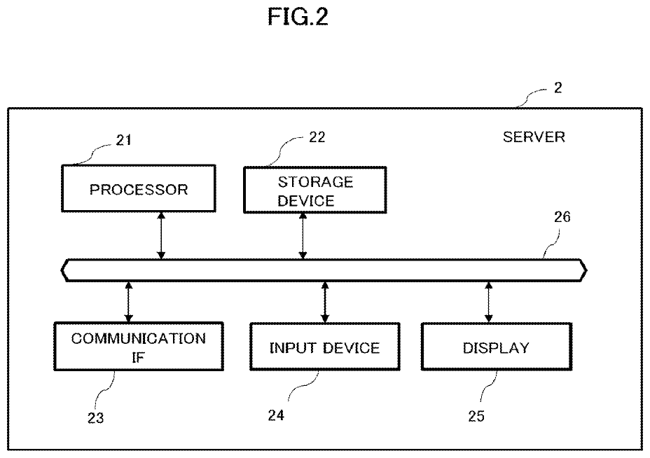

FIG. 2 is a diagram illustrating an example of the structure of the server 2. The server 2 may include a dedicated or general purpose information processing apparatus (computer) such as a personal computer (PC), a workstation (WS), or a server machine. The server 2 can communicate with the terminal 3 and the terminal 4 via the network 1 using a communication function. The connection to the network 1 may be wired or wireless. The server 2 may include one information processing apparatus or a set (cloud) of two or more information processing apparatus. The server 2 may be a fixed terminal or a mobile terminal.

The server 2 includes a processor 21 as a processing unit or the control unit (controller), a storage device 22, a communication interface 23 (communication IF 23), an input device 24, and a display 25 that are connected to each other via a bus 26.

The storage device 22 includes a main storage device and an auxiliary storage device. The main storage device is used as at least one of a storage area for programs and data, an expansion area for programs, a work area for programs, a buffer area for communication data, and the like. The main storage device includes, for example, a random access memory (RAM) or a combination of a RAM and a read only memory (ROM). The auxiliary storage device is used as a storage area for data and programs. A non-volatile storage medium is used as the auxiliary the storage device. The non-volatile storage medium is, for example, a hard disk drive, a solid state drive (SSD), a flash memory, or an EEPROM (electrically erasable programmable read-only memory). The storage device 22 can also include a drive device for a disc recording medium.

The communication IF 23 is a circuit that performs communication processing. For example, the communication IF 23 is a network interface card (NIC). Alternatively, the communication IF 23 may be a wireless communication circuit that performs wireless communication (such as LTE, 5G, wireless LAN (Wi-Fi), BLE). Alternatively, the communication IF 23 may be a combination of a circuit for wired communication processing and a wireless communication circuit.

The input device 24 includes keys, buttons, a pointing device, a touch panel, and the like and is used to input information. The display 25 is, for example, a liquid crystal display and displays information and data.

The processor 21 is, for example, a central processing unit (CPU). The CPU is also referred to as a microprocessor unit (MPU). The processor 21 may have a single-processor structure or a multi-processor structure. Alternatively, a single physical CPU connected via a single socket may have a multi-core structure. The processor 21 may include an arithmetic unit of various circuit structures such as a digital signal processor (DSP) or a graphics processing unit (GPU). Alternatively, the processor 21 may have a structure that cooperates with at least one of an integrated circuit (IC), another digital circuit, an analog circuit, and the like. The integrated circuit includes an LSI, an application specific integrated circuit (ASIC), a programmable logic device (PLD), and the like. The PLD includes, for example, a field-programmable gate array (FPGA). The processor 21 also includes what is called, for example, a microcontroller (MCU), a SoC (system-on-a-chip), a system LSI, a chipset, or the like.

The processor 21 performs various types of processing by executing various programs stored in the storage device 22.

Table Structure

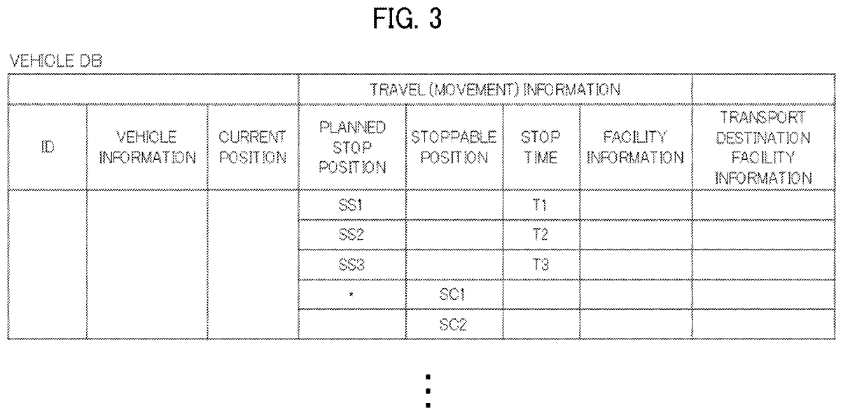

FIG. 3 illustrates an example of the data structure of a vehicle database (vehicle DB) stored in the storage device 22 of the server 2. The vehicle DB is used to manage information about the railroad vehicle for each railroad vehicle (composition) to be managed. FIG. 3 illustrates the information elements retained for one railroad vehicle (railroad vehicle 10 with an identifier (ID) of V1).

In FIG. 3, the vehicle DB can store the vehicle information, the current position, the travel information, and the transport destination facility information about the railroad vehicle 10 (with an ID of V1). The ID is the identifier of the railroad vehicle 10. The vehicle information indicates information indicating the composition of the railroad vehicle 10 (the number of vehicles, the starting station, the stops, and the end point, the scheduled arrival and departure times of stations) and the like.

The current position is information indicating the current position of the terminal 3 received from the terminal 3 or the current position converted from the travel distance measured by the encoder of the railroad vehicle 10.

The travel information includes information indicating the planned stop positions, the stoppable position, and the stop times. The travel information may further include the facility information of the planned stop positions or the stoppable position. The planned stop positions and the stoppable position are information indicating the planned stop positions SS1, SS2, and SS3, the stoppable position SC1, and the like illustrated in FIG. 1. The stop times are information indicating the planned arrival and departure times at the planned stop positions. However, the stoppable position does not have information about the stop time because it indicates the stop position in an emergency. When the use of the stoppable position is determined, the planned arrival time at the stoppable position is calculated by the processor 21 or the like.

The facility information is information about the facility to which the planned stop position or the stoppable position belongs. The facility is a station, a garage, or a base and the facility information may include information indicating rescue actions that can be taken by the rescuer at the facility. The facility information includes information indicating the access methods (transporters) that the persons 12 can use for the facility. For example, when there a parking space for vehicles (private vehicles) or the like of the persons 12, access by vehicles is permitted. When there is no parking space, the persons 12 makes access by public transports or walking.

Also, the transport destination facility information may be stored in association with the planned stop position or the stoppable position. The transport destination facility information indicates the name, the address (position), the contact information, and the like of the facility (such as a hospital, a clinic, an osteopathic clinic) to which the rescuee is transported. Since the person 11 can safely get off the railroad vehicle 10 in the planned stop position and the stoppable position, the planned stop position or the stoppable position is the meeting point at which the rescuee meets the rescuer in the embodiment.

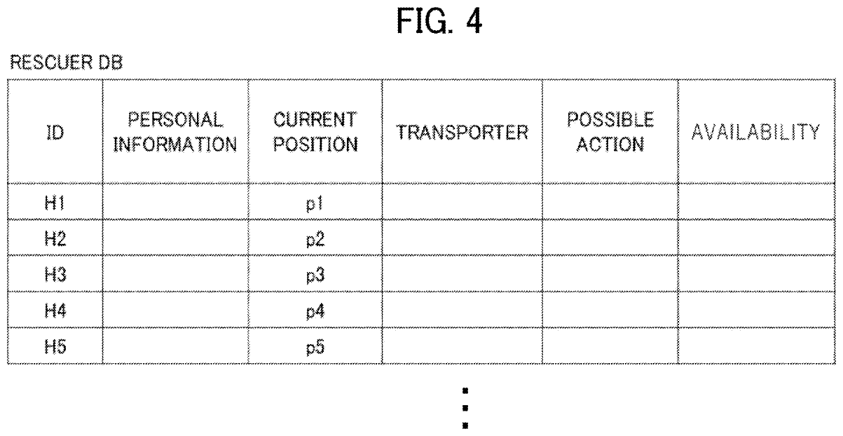

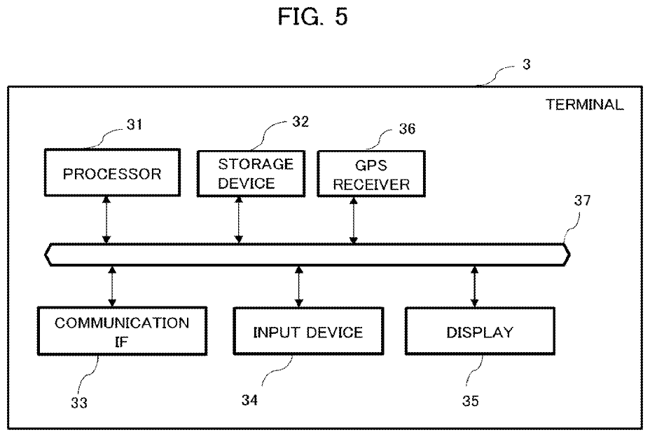

FIG. 4 illustrates an example of the data structure of a rescuer database (rescuer DB) stored in the storage device 22 of the server 2. The rescuer DB has a table structure and includes a plurality of records prepared for the persons 12 registered as the rescuers. The record stores information indicating the ID (identifier), the personal information, the current position, the transporter, the possible actions, and the availability of the rescuer. For example, the persons 12 registers the information in the rescuer DB via the corresponding terminal 4 in advance by accessing the server 2. The position information of the terminals 4 is periodically transmitted to the server 2 and the current positions in the rescuer DB are updated.

The personal information includes the name, the sex, the age, the contact information (address, telephone number, or e-mail address), the network address of the terminal 4 of the person 12 who is the rescuer, and the like. The positions of the terminals 4 received from the terminals 4 are managed as the current positions of the persons 12. The information indicating the transporter includes information that indicates the transporter (walking, bicycle, private car, or public transportation) used by the person 12 to travel to the meeting point as the rescuer and the time required to travel to the meeting point. The possible actions indicate the types of actions that the person 12 can perform on the rescuee. The actions include watching, attendance (assisting of walking or traveling), treating minor injury, emergency lifesaving, and medical actions that can be performed by a nurse or a paramedic, and medical actions that can be performed only by a doctor.

The availability indicates whether a call for service as the rescuer can be accepted at that time. The state (OK or NG) indicating the availability can be changed as appropriate by the person 12 using the terminal 4.

Structure of the Terminal

FIG. 4 illustrates an example of the structure of a terminal device 30 (referred to below as a terminal 30) capable of operating as the terminal 3 or the terminal 4. The terminal 30 is, for example, a mobile terminal having a wireless communication function (portable terminal: a terminal having portability). The mobile terminal is, for example, a smartphone, a tablet terminal, a laptop personal computer (PC), a personal digital assistant (PDA), or a wearable computer. However, the terminal 3 may be a fixed terminal. In addition, the terminal 30 may be an in-vehicle terminal mounted on the railroad vehicle 10. The terminal 30 used as the terminal 4 may be a fixed terminal.

The terminal 30 includes a processor 31, a storage device 32, a communication interface (communication IF) 33, an input device 34, a display 35, and a GPS receiver 36 connected to each other via a bus 37.

The components similar to those described as the processor 21, the storage device 22, the communication IF 23, the input device 24, and the display 25 are applicable to the processor 31, the storage device 32, the communication IF 33, the input device 34, and the display 35, respectively. However, depending on differences in the use and the purpose of use and the like, the components with different performance from the components applied to the server 2 are applied.

The GPS receiver 36 receives the radio waves from GPS satellites and detects the position of the terminal 30. The processor 31 performs various types of processing by executing various program stored in the storage device 32.

Example of Operation

### Processing in the Terminal 3

In the terminal 3 (the terminal 30 operating as the terminal 3), the processor 31 executes a program stored in the storage device 32. When the program is executed, the terminal 3 performs processing for transmitting, to the server 2, a message for a rescue request for asking the rescue of the person 11. In addition, the processor 31 performs processing for outputting (displaying) the rescuer and the meeting point included in the message for notification received from the server 2. The processing related to the generation and transmission of the rescue request and the display of the rescuer and the meeting point is performed by use of a web browser or a dedicated application program (application) installed in the terminal 3.

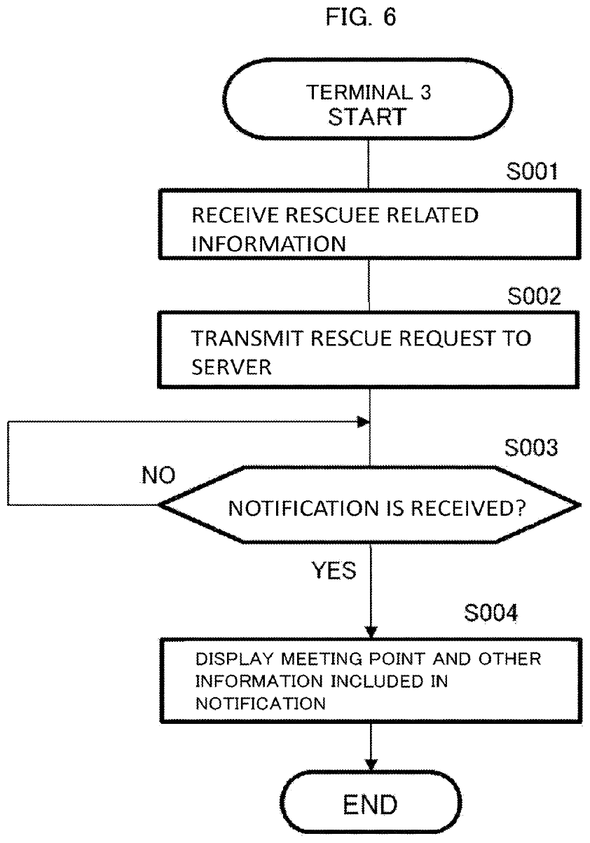

FIG. 6 is a flowchart illustrating an example of processing by the terminal 3. In step S001, the processor 31 of the terminal 3 receives the rescuee related information input to the terminal 3 via the input device 34. The user of the terminal 3 is, for example, the conductor or the driver of the railroad vehicle 10, but the user may be the person 11. The user may be other than the conductor, the driver, and the person 11.

The rescuee related information relates to the person 11 as the rescuee. The rescuee related information includes information indicating the car number, the name, the sex, the age, the blood type, the clothes, the characteristics, the conditions (such as medical condition, symptom, and the degree of injury) of the person 11, the required actions by the rescuer, the number of rescuers, and the like. However, the rescuee related information may be only the information (such as the sex, the clothes, the predicted age, the symptoms) that can be obtained by appearances among the information described above because the person 11 may have lost consciousness.

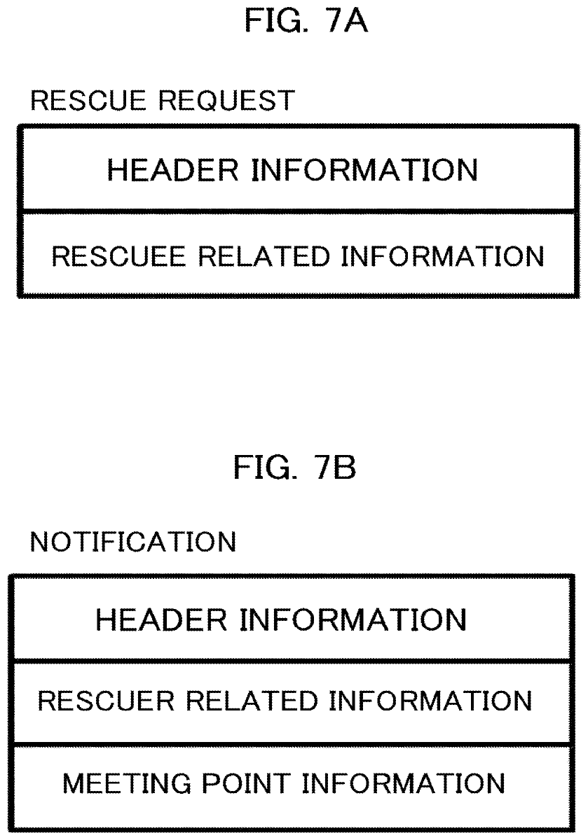

In step S002, the processor 31 performs processing for generating a message for a rescue request and transmitting the message to the server 2. FIG. 7A illustrates an example of the format of the message for a rescue request. The rescue request includes the header information and the rescuee related information. The header information includes the message ID, the message type, the transmission destination address (address of the server 2, known), the transmission source address (the network address of terminal 3), the application number (the identification number of the application that processes the message for an emergency request), and the like.

In step S003, the processor 31 enters the state that waits for the message for notification from the server 2. When the notification is received, the processing proceeds to step S004. In step S004, the processor 31 performs processing for displaying (outputting), in the display 35, the meeting point and the rescuer related information included in the notification.

As illustrated in FIG. 7B, the message for notification includes the header information, the rescuer related information, and the meeting point information. The header information includes the message ID, the transmission destination address (the network address of the terminal 3), the transmission source address (the network address of the server 2), the application number, and the like included in the rescue request. The rescuer related information indicates the characteristics such as the name, the affiliation, the contact information, and the clothes of the rescuer. The meeting point information indicates the planned stop position (the station name and the platform number), the waiting place (for example, the vicinity of the stop position of the number of the car on which the rescuee rides) of the rescuer.

Processing by the Server 2

In the server 2, the processor 21 executes the program stored in the storage device 22. When the program is executed, the server 2 performs processing with respect to the message for a rescue request from the terminal 3 and performs processing for transmitting a notification corresponding to the rescue request to the terminal 3 and the terminal 4 of the rescuer.

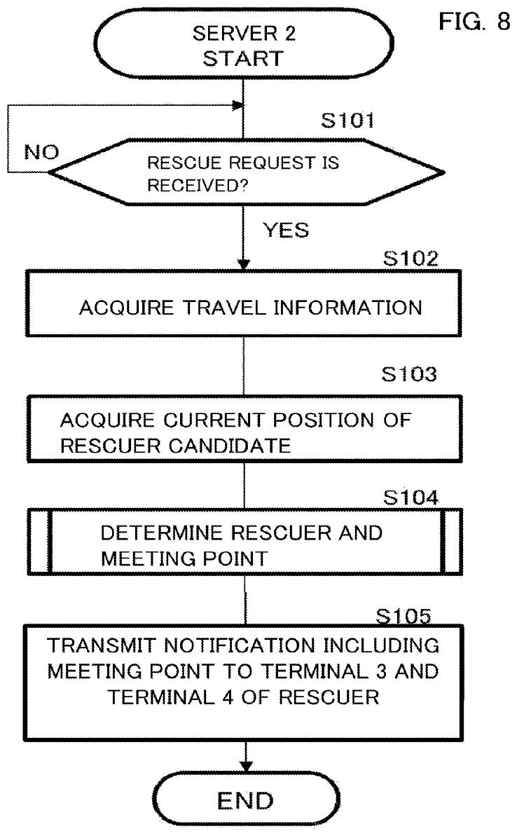

FIG. 8 is a flowchart illustrating an example of processing by the server 2. In step S101, the processor 21 of the server 2 waits for a message for inquiry and, when determining that the message has been received, the processing proceeds to step S102.

In step S102, the processor 21 reads (acquires) the travel information of the railroad vehicle 10 from the vehicle DB with reference to the vehicle DB (FIG. 3) stored in the storage device 22. In step S103, the processor 21 reads (acquires) the current positions of the rescuer candidates from the rescuer DB with reference to the rescuer DB (FIG. 4) stored in the storage device 22. However, the current position information of the persons 12 whose availability states are “NG” is excluded from the acquisition target (not the rescuer candidates).

In step S104, the processor 21 determines the rescuer and the meeting point using the travel information acquired in step S102 and the current positions of the rescuer candidates (persons 12) acquired in step S103.

In step S105, the processor 21 generates a message for the notification including information indicating the meeting point, and transmits the message for notification to terminal 3 and the terminal 4 of the rescuer.

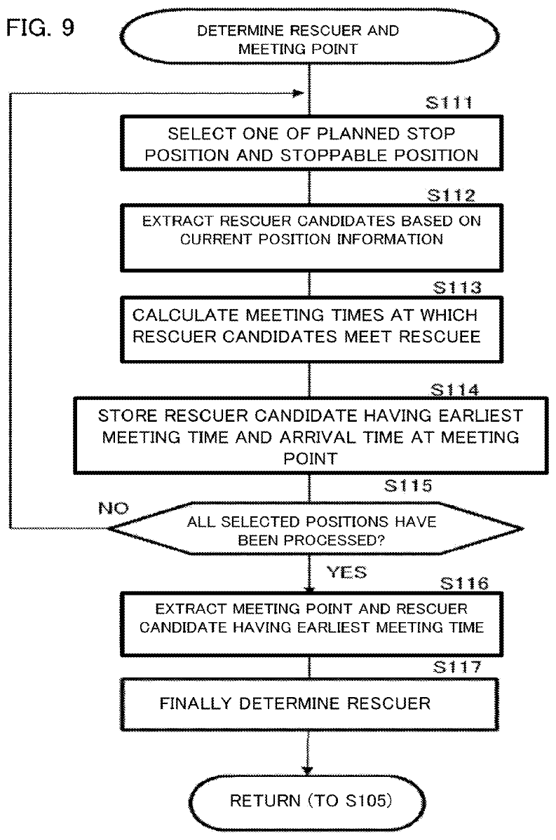

FIG. 9 is a flowchart illustrating an example of a subroutine of determination processing (step S104) for the rescuer and the meeting point. In step S111, the processor 21 selects any one of the planned stop position and the stoppable position included in the travel information. In the following description, this position is referred to as the “selected position”.

In step S112, the processor 21 extracts the rescuer candidates based on the current position information. That is, the processor 21 extracts the persons 12 who are present in the predetermined geographical range from the selected position as the rescuer candidates among the current positions of the persons 12 (terminals 4) obtained from the rescuer DB. The predetermined geographical range can be specified so that, for example, the current positions of persons 12 are within a circle having a predetermined radius centered on the coordinates of the selected position. The figure centered on the selected position may be an ellipse, a triangle, or a polygon having four or more sides instead of a circle. It should be noted that the persons 12 for whom the distances between the current positions and the positions selected in step S111 are within a predetermined distance may be extracted as the rescuer candidates. When the persons 12 are not present, the terminal 3 is notified of error.

In step S113, the processor 21 calculates the time (meeting time) at which the rescuer candidate can meet the rescuee at the selected position for each of the rescuer candidates (persons 12) extracted in step S112 when the selected position is assumed to be the meeting point. In calculating the arrival time, the selected position of each of the rescuer candidates, the average speed predetermined for each transporter can be used as the travel speed of the rescuer candidate. The transporter having the fastest travel speed is selected as the transporter of the rescuer candidate from the transporters, used by the rescuer candidates, that are permitted to be used at the facility to which the selected position belongs. For example, when the transporters that can be used by the rescuer candidate are a vehicle and walking, the speed of a vehicle faster than a walk is selected as the transport speed of the rescuer candidate.

The processor 21 calculates the required travel time for each of the rescuer candidates using the distance between the current position and the selected position and the travel speed of the transporter and calculates the planned arrival time at the selected position. In addition, the processor 21 calculates the time at which each of the rescuer candidates meets the rescuee by using the planned arrival time of the railroad vehicle 10 at the selected position. Then, the rescuer is determined among the rescuer candidates in order of the meeting time from earliest to latest. For example, the rescuer candidate with the earliest meeting time may be determined as the rescuer.

Here, when the arrival time of the rescuer candidate at the selected position is earlier than the planned arrival time of the railroad vehicle 10, the planned arrival time of the railroad vehicle 10 is treated as the meeting time. In contrast, when the arrival time of the rescuer candidate at the selected position is later than the planned arrival time of the railroad vehicle 10, the arrival time of the rescuer candidate at the selected position is treated as the meeting time. As described above, the rankings of the persons 12 who can arrive at the selected position before the railroad vehicle 10 arrives are treated as the same ranking. This is because there is no superiority or inferiority in that all of the persons 12 can wait at the station and the like.

In step S115, the processor 21 determines whether the processing of steps S112 to S114 has been completed for all the selected positions. At this time, when it is determined that the processing of steps S112 to S114 has been completed for all the selected positions, the processing proceeds to step S116. In contrast, when it is determined that the processing has not been completed in step S115, the processing returns to step S111, another the selected position is selected, and processing of steps S112 to S114 is performed.

When the processing proceeds to step S116, the processor 21 determines the selected position having the earliest meeting time as the meeting point and extracts the rescuer candidate with the earliest meeting time at the meeting point.

In step S117, a final decision of the rescuer is made. When the number of the applied rescuer candidates is the same as the number (capacity) of the rescuers specified in the rescue request in step S116, these rescuer candidates are determined to be the rescuers. In contrast, when the number of the rescuer candidates exceeds the number of the rescuers, the specified number of the rescuer candidates is determined to be the rescuers by an appropriate method.

For example, the specified number of the rescuer candidates may be determined to be the rescuers in order of arrival at the meeting point. Alternatively, the processor 21 may preferentially select the rescuer candidates of the same sex as the rescuee. Alternatively, the processor 21 may extract the specified number of rescuer candidates capable of performing necessary actions based on the information indicating actions (necessary actions) to be performed on the rescuee and the information indicating actions that can be performed by the plurality of rescuer candidates, and may determine the extracted rescuer candidates as the rescuers. The rescuer candidates to be finally determined as the rescuers can be set as appropriate. Upon completion of the processing of step S117, the processing proceeds to step S105.

It should be noted that the processor 21 may refer to the transport destination facility information when the rescuee request transportation to a facility such as a hospital in determining the meeting point. Then, the processor 21 may calculate the distance between the planned stop position or the stoppable position and the destination facility using the transport destination facility information and preferentially determine the planned stop position or the stoppable position having the shortest distance as the meeting point.

Processing by the Terminal 4

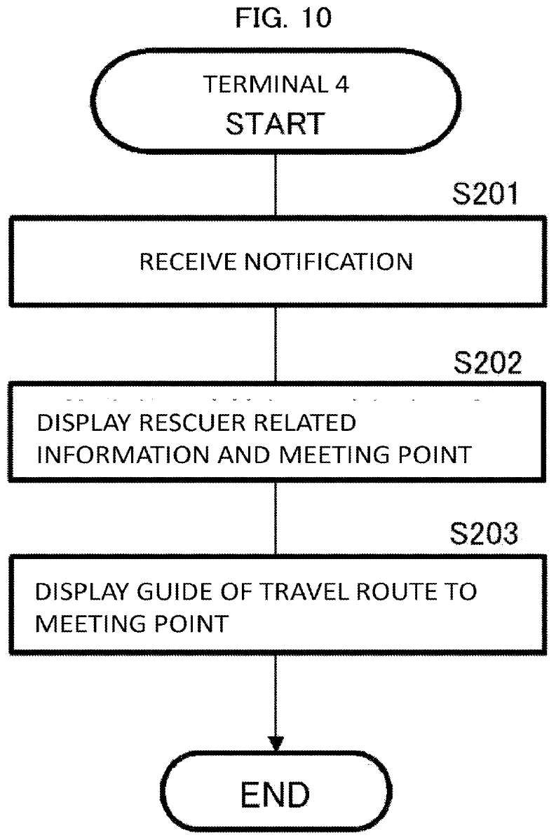

FIG. 10 is a flowchart illustrating an example of processing by the terminal 30 that operates as the terminal 4 of the rescuer. In step S201, the processor 31 receives a message for notification from the server 2. In step S202, the processor 31 performs processing for displaying the rescuer related information and the meeting point information on the display 35. In step S203, the processor 31 calculates the travel route from the current position to the meeting point and displays information that guides the person as the rescuer to the meeting point on the display 35. The person 12 as the rescuer can come to rescue the rescuee with reference to the displayed information.

### Effect of the Embodiment

The server 2 as the information processing apparatus according to the embodiment determines the rescuer and the meeting point based on the travel information and the position information of the rescuer candidates. This can determine the meeting point and the rescuer that are suitable for the rescuee who is traveling on the railroad vehicle 10 according to the operation schedule of the railroad vehicle 10 (the planned travel route and the travel schedule). In addition, since the server 2 determines the planned stop position or the stoppable position at which the rescuer can meet the rescuee in the shortest time period as the meeting point, so the rescuer can rescue the rescuee as soon as possible. In addition, when a number of rescuer candidates more than a specified number are selected, the server 2 determines preferable persons as the rescuers.

Others

The embodiment described above is only an example and the present disclosure can be implemented by making changes as appropriate without departing from the spirit.

In addition, the processing described as being performed by one device may be performed by a plurality of devices in a shared manner. Alternatively, the processing described as being performed by different devices may be performed by one device. In the computer system, the hardware structures (server structures) for achieving the individual functions can be flexibly changed.

The present disclosure can also be achieved by supplying computer programs that implement the functions described in the embodiment to a computer and causing one or more processors of the computer to read and execute the programs. Such computer programs may be provided for the computer by a non-transitory computer readable storage medium that can be connected to the system bus of the computer or may be provided for the computer via a network. The non-transitory computer readable storage media is a disk of any type, a magnetic disk (such as a floppy (registered trademark) disk or a hard disk drive (HDD)), an optical disk (such as a CD-ROM, a DVD disk, a Blu-ray disk), or the like. In addition, the non-transitory computer readable medium includes a read-only memory (ROM), a random access memory (RAM), an EPROM, an EEPROM, a magnetic card, a flash memory, an optical card, or a medium of any type suitable for storing electronic instructions.

What is claimed is:

1. An information processing apparatus comprising:
a controller configured to:
acquire travel information including a planned travel route and a travel schedule of a moving body on which a rescuee rides;
acquire position information of one or more rescuer candidates capable of rescuing the rescue; and
determine a rescuer among the one or more rescuer candidates based on the position information of the one or more rescuer candidates and the travel information and determines a meeting point at which the rescuer should meet the rescuee.

2. The information processing apparatus according to claim 1, wherein the controller is configured to select the rescuer from the one or more rescuer candidates who are present in a predetermined geographical range from the planned travel route of the moving body.

3. The information processing apparatus according to claim 1, wherein the controller is configured to calculate a point and a time at which each of the rescuer candidates is able to meet the rescuee in a shortest time period based on the travel information and the position information of each of the rescuer candidates and determines the rescuer in order of the time from earliest to latest.

4. The information processing apparatus according to claim 3, wherein, when there are a plurality of rescuer candidates for whom the time at which the rescuer candidates are able to meet the rescuee is the earliest, the controller is configured to determine the rescuer from among the plurality of rescuer candidates by preferentially selecting the rescuer candidates of the same sex as the rescuee.

5. The information processing apparatus according to claim 3, wherein, when there are a plurality of rescuer candidates for whom the time at which the rescuer candidates are able to meet the rescuee is the earliest, the controller is configured to determine the rescuer based on information indicating an action to be performed on the rescuee and information indicating an action that each of the plurality of rescuer candidates is able to perform on the rescuee.

6. The information processing apparatus according to claim 1, wherein the controller is configured to extract one or more points in the planned travel route at which the moving body is able to stop and determines the meeting point from among the extracted points.

7. The information processing apparatus according to claim 1, wherein the controller is configured to notify the moving body and the rescuer of information indicating the meeting point.

8. An information processing method comprising:
acquiring, by an information processing apparatus, travel information that includes a planned travel route and a travel schedule of a moving body on which a rescuee rides;
acquiring, by an information processing apparatus, position information of one or more rescuer candidates capable of rescuing the rescuee; and
determining, by an information processing apparatus, a rescuer from among the one or more rescuer candidates based on the position information of the one or more rescuer candidates and the travel information and determines a meeting point at which the rescuer meet should the rescuee.

9. The information processing method according to claim 8, wherein the information processing apparatus selects the rescuer from the one or more rescuer candidates who are present in a predetermined geographical range from the planned travel route of the moving body.

10. The information processing method according to claim 8, wherein the controller calculates a point and a time at which each of the rescuer candidates meets the rescuee in a shortest time period based on the travel information and the position information of each of the rescuer candidates and determines the rescuer in order of the time from earliest to latest.

11. The information processing method according to claim 10, wherein, when there are a plurality of rescuer candidates for whom the time at which the rescuer candidates are able to meet the rescuee is the earliest, the information processing apparatus determines the rescuer from among the plurality of rescuer candidates by preferentially selecting the rescuer candidates of the same sex as the rescuee.

12. The information processing method according to claim 10, wherein, when there are a plurality of rescuer candidates for whom the time at which the rescuer candidates are able to meet the rescuee is the earliest, the information processing apparatus determines the rescuer based on information indicating an action to be performed on the rescuee and information indicating an action that each of the plurality of rescuer candidates are able to perform on the rescuee.

13. The information processing method according to claim 9, wherein the information processing apparatus extracts one or more points in the planned travel route at which the moving body is able to stop and determines the meeting point from among the extracted points.

14. The information processing method according to claim 8, wherein the information processing apparatus notifies the moving body and the rescuer of the meeting point.

15. A non-transitory storage medium storing a program causing a computer to:
acquire travel information that includes a planned travel route and a travel schedule of a moving body on which a rescuee rides;
acquire position information of each of one or more rescuer candidates capable of rescuing the rescue; and
determine a rescuer from among the one or more rescuer candidates based on the position information of each of the rescuer candidates and the travel information and determine a meeting point at which the rescuer should meet the rescuee.

16. The non-transitory storage medium according to claim 15, wherein the program causes the computer to select the rescuer from the rescuer candidates who are present in a predetermined geographical range from the planned travel route of the moving body.

17. The non-transitory storage medium according to claim 15, wherein the program causes the computer to calculate a point and a time at which each of the rescuer candidates meets the rescuee in a shortest time period based on the travel information and the position information of each of the rescuer candidates and determines the rescuer in order of the time from earliest to latest.

18. The non-transitory storage medium according to claim 17, wherein, when there are a plurality of rescuer candidates for whom the time at which the rescuer candidates meet the rescuee is the earliest, the program causes the computer to determine the rescuer based on information indicating an action to be performed on the rescuee and information indicating an action that each of the plurality of rescuer candidates is able to perform on the rescuee.

19. The non-transitory storage medium according to claim 15, wherein the program causes the computer to extract one or more points in the planned travel route at which the moving body is able to stop and determine the meeting point among the extracted points.

20. The non-transitory storage medium according to claim 15, wherein the program causes the computer to notify the moving body and the rescuer of the meeting point.
